(19) United States
(12) Patent Application Publication
(10) Pub. No.: US 2005/0221318 A1
(43) Pub. Date: Oct. 6, 2005

(54) Building block forming a c-c bond upon reaction

(76) Inventors: Alex Haahr Gouliaev, Veksoe Sjaelland (DK); Henrik Pedersen, Bagsvaerd (DK); Kim Birkebaek Jensen, Roedovre (DK); Anders Holm Hansen, Copenhagen NV (DK); Christian Sams, Frederiksberg C (DK); Jakob Felding, Charlottelund (DK)

Correspondence Address: BROWDY AND NEIMARK, P.L.L.C.;624 NINTH STREET, NW, SUITE 300, WASHINGTON, DC, 20001-5303, US

(21) Appl. No.: 10/507,599

(22) PCT Filed: Mar. 14, 2003
(86) PCT No.: PCT/DK03/00175

§ 371 Date: Apr. 7, 2005

(30) Foreign Application Priority Data
Mar. 15, 2002 (DK) PA 2002 0415
Mar. 15, 2002 (US) 60364056
Dec. 19, 2002 (DK) PA 2002 01947
Dec. 19, 2002 (US) 60434428

(51) Int. Cl. C12Q 1/68; G01N 33/53

(52) U.S. Cl. 435/6; 435/7.1

(57) ABSTRACT

A building block having the dual capabilities of recognising an encoding element and transferring a functional entity to a recipient reactive group is disclosed. The building block may be used in the generation of a single complex or libraries of different complexes, wherein the complex comprises an encoded molecule linked to an encoding element. Libraries of complexes are useful in the quest for pharmaceutically active compounds.

### TECHNICAL FIELD OF THE INVENTION

The present invention relates to a building block comprising a complementing element and a precursor for a functional entity. The building block is designed to transfer the functional entity to a recipient reactive group upon recognition between the complementing element and an encoding element associated with the reactive group.

### BACKGROUND

The transfer of a chemical entity from one mono-, di- or oligonucleotide to another has been considered in the prior art. Thus, N. M. Chung et al. (Biochim. Biophys. Acta, 1971, 228, 536-543) used a poly(U) template to catalyse the transfer of an acetyl group from 3′-O-acetyladenosine to the 5′-OH of adenosine. The reverse transfer, i.e. the transfer of the acetyl group from a 5′-O-acetyladenosine to a 3′-OH group of another adenosine, was also demonstrated.

Walder et al. Proc. Natl. Acad. Sci. USA, 1979, 76, 51-55 suggest a synthetic procedure for peptide synthesis. The synthesis involves the transfer of nascent immobilized polypeptide attached to an oligonucleotide strand to a precursor amino acid attached to an oligonucleotide. The transfer comprises the chemical attack of the amino group of the amino acid precursor on the substitution labile peptidyl ester, which in turn results in an acyl transfer. It is suggested to attach the amino acid precursor to the 5′ end of an oligonucleotide with a thiol ester linkage.

The transfer of a peptide from one oligonucleotide to another using a template is disclosed in Bruick RK et al. Chemistry & Biology, 1996, 3:49-56. The carboxy terminal of the peptide is initially converted to a thioester group and subsequently transformed to an activated thioester upon incubation with Ellman's reagent. The activated thioester is reacted with a first oligo, which is 5′-thiol-terminated, resulting in the formation of a thio-ester linked intermediate. The first oligonucleotide and a second oligonucleotide having a 3′ amino group is aligned on a template such that the thioester group and the amino group are positioned in close proximity and a transfer is effected resulting in a coupling of the peptide to the second oligonucleotide through an amide bond.

### SUMMARY OF THE INVENTION

The present invention relates to a building block of the general formula:
Complementing Element-Linker-Carrier-Functional entity precursor
capable of transferring a functional entity to a recipient reactive group, wherein
Complementing Element is a group identifying the functional entity,
Linker is a chemical moiety comprising a spacer and a S—C-connecting group, wherein the spacer is a valence bond or a group distancing the functional entity precursor to be transferred from the complementing element and the S—C-connecting group connects the spacer with the Carrier,
Carrier comprises an aromatic-, a saturated- or a partially saturated heterocyclic ring system, said ring system being mono, di- or tricyclic and substituted with 0-3 R¹ and containing a ring-member M belonging to the group consisting of B, Si, Sn and Zn, whereas M carries the functional entity precursor and 0-2 ligands L selected independently from the group consisting of —F, -aryl, -heteroaryl, or
Carrier is —Ar-M(L)ₚ—, —Ar-(C₁-C₆ alkylene)-M(L)ₚ— or —Ar—X—(C₁-C₆ alkylene)M(L)ₚ— where Ar is aryl or heteroaryl substituted with 0-3 R¹, M is B, Sn or Si, X is O, S, or R² and L is independently chosen from —F, -aryl, -heteroaryl or C₁-C₆ alkyl; R¹ and R¹, are independently selected from —H, —OR², —NR²₂, -Halogen, —NO₂, —CN, —C(Halogen)₃, —C(O)R², —C(O)NHR², C(O)NR²₂, —NC(O)R², —S(O)₂NHR², —S(O)₂NR²₂, —S(O)₂R², —P(O)₂—R², —P(O)—R², —S(O)—R², P(O)—OR², —S(O)—OR², —N⁺R²₃, wherein p is an integer of 0 to 3 and R² is H, C₁-C₆ alkyl, C₂-C₆ alkenyl, C₂-C₈ alkynyl, or aryl,
Functional entity precursor is H or selected among the group consisting of a C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R³, 0-3 R⁴ and 0-3 R⁷ or C₁-C₃ alkylene-NR³₂, C₁-C₃ alkylene-NR³C(O)R⁶, C₁-C₃ alkylene-NR³C(O)OR⁶, C₁-C₂ alkylene-O—NR³₂, C₁-C₂ alkylene-O—NR³C(O)R⁶, C₁-C₂ alkylene-O—NR³C(O)OR⁶ substituted with 0-3 R⁷.
where R³ is H or selected independently among the group consisting of C₁-C₆ alkyl, C₂-C₆ alkenyl, C₂-C₈ alkynyl, C₃-C₇ cycloalkyl, C₃-C₇ cycloheteroalkyl, aryl, heteroaryl, said group being substituted with 0-3 R⁴ and 0-3 R⁷ and
R⁴ is selected independently from —N₃, —CNO, —C(NOH)NH₂, —NHOH, —NHNH, —C(O), —P(O)(O)₂ or the group consisting of C₂-C₆ alkenyl, C₂-C₆ alkynyl, C₄-C₈ alkadienyl said group being substituted with 0-2 R⁵,
where R⁵ is independently selected from —NO₂, —C(O)O, —C(O), —CN, —Si₃, —O and —N₂.
R⁶ is H, C₁-C₆ alkyl, C₂-C₆ alkenyl, C₂-C₆ alkynyl, C₃-C₇ cycloalkyl, aryl or C₁-C₆ alkylene-aryl substituted with 0-3 substituents independently selected from —F, —Cl, —NO₂, —R², —OR², —SiR²₃
   R⁷ is ═O, —F, —Cl, —Br, —I, —CN, —NO₂, —O, —N₂, —N—C(O)R⁶, —N—C(O)OR⁶, —S, —S(O), —S(O)₂, —COO, —C(O)N₂, or —S(O)₂N₂,

In the following description of the invention the direction of connections between the various components of a building block should be read left to right. For example an S—C-connecting group C(═O)—NH— is connected to a Spacer through the carbon atom on the left and to a Carrier through the nitrogen atom on the right hand side.

The term “C₃-C₇ cycloheteroalkyl” as used herein refers to a radical of totally saturated heterocycle like a cyclic hydrocarbon containing one or more heteroatoms selected from nitrogen, oxygen, phosphor, boron and sulphur independently in the cycle such as pyrrolidine (1-pyrrolidine; 2-pyrrolidine; 3-pyrrolidine; 4-pyrrolidine; 5-pyrrolidine); pyrazolidine (1-pyrazolidine; 2-pyrazolidine; 3-pyrazolidine; 4-pyrazolidine; 5-pyrazolidine); imidazolidine (1-imidazolidine; 2-imidazolidine; 3-imidazolidine; 4-imidazolidine; 5-imidazolidine); thiazolidine (2-thiazolidine; 3-thiazolidine; 4-thiazolidine; 5-thiazolidine); piperidine (1-piperidine; 2-piperidine; 3-piperidine; 4-piperidine; 5-piperidine; 6-piperidine); piperazine (1-piperazine; 2-piperazine; 3-piperazine; 4-piperazine; 5-piperazine; 6-piperazine); morpholine (2-morpholine; 3-morpholine; 4-morpholine; 5-morpholine; 6-morpholine); thiomorpholine (2-thiomorpholine; 3-thiomorpholine; 4-thiomorpholine; 5-thiomorpholine; 6-thiomorpholine); 1,2-oxathiolane (3-(1,2-oxathiolane); 4-(1,2-oxathiolane); 5-(1,2-oxathiolane); 1,3-dioxolane (2-(1,3-dioxolane); 4-(1,3-dioxolane); 5-(1,3-dioxolane); tetrahydropyrane; (2-tetrahydropyrane; 3-tetrahydropyrane; 4-tetrahydropyrane; 5-tetrahydropyrane; 6-tetrahydropyrane); hexahydropyridazine (1-(hexahydropyridazine); 2-(hexahydropyridazine); 3-(hexahydropyridazine); 4-(hexahydropyridazine); 5-(hexahydropyridazine); 6-(hexahydropyridazine)), [1,3,2]dioxaborolane, [1,3,6,2]dioxazaborocane.

The term “aryl” as used herein includes carbocyclic aromatic ring systems of 5-7 carbon atoms. Aryl is also intended to include the partially hydrogenated derivatives of the carbocyclic systems as well as up to four fused aromatic- or partially hydrogenated rings, each ring comprising 5-7 carbon atoms.

The term “heteroaryl” as used herein includes heterocyclic unsaturated ring systems containing, in addition to 2-18 carbon atoms, one or more heteroatoms selected from nitrogen, oxygen and sulphur such as furyl, thienyl, pyrrolyl, heteroaryl is also intended to include the partially hydrogenated derivatives of the heterocyclic systems enumerated below.

The terms “aryl” and “heteroaryl” as used herein refers to an aryl which can be optionally substituted or a heteroaryl which can be optionally substituted and includes phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydrobenzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydrobenzo[b]thiophenyl), 5-(2,3-dihydro-benzo[b]thiophenyl), 6-(2,3-dihydrobenzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl).

The Functional Entity carries elements used to interact with host molecules and optionally reactive elements allowing further elaboration of an encoded molecule of a library. Interaction with host molecules like enzymes, receptors and polymers is typically mediated through van der waal's interactions, polar- and ionic interactions and pi-stacking effects. Substituents mediating said effects may be masked by methods known to an individual skilled in the art (Greene, T. W.; Wuts, P. G. M. Protective Groups in Organic Synthesis; 3rd ed.; John Wiley & Sons: New York, 1999.) to avoid undesired interactions or reactions during the preparation of the individual building blocks and during library synthesis. Analogously, reactive elements may be masked by suitably selected protection groups. It is appreciated by one skilled in the art that by suitable protection, a functional entity may carry a wide range of substitutents.

The Functional Entity Precursor is a masked Functional Entity that is incorporated into an encoded molecule. After incorporation, reactive elements of the Functional Entity may be revealed by un-masking allowing further synthetic operations. Finally, elements mediating recognition of host molecules may be un-masked.

The function of the carrier is to ensure the transferability of the functional entity. To adjust the transferability a skilled chemist can design suitable substitutions of the carrier by evaluation of initial attempts. The transferability may be adjusted in response to the chemical composition of the functional entity, to the nature of the complementing element, to the conditions under which the transfer and recognition is performed, etc.

In a preferred embodiment, the carrier is selected from the group consisting of: 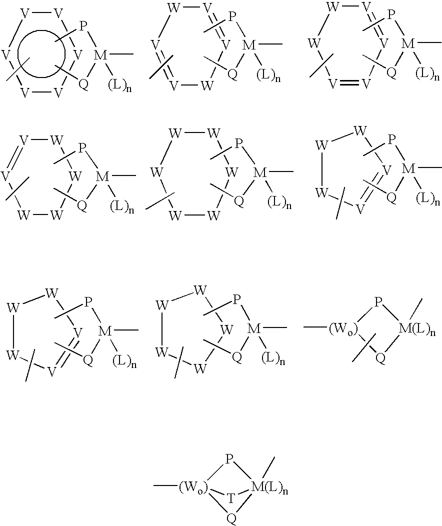
wherein
W is —O—, —S—, —CR¹R¹—, —C(═O)—, —C(═S)—, —C(═NR²)— or —NR¹—;
V is —N═, —CR¹═;
P, Q and T are independently absent or are independently chosen from —CR¹R¹—, —NR¹—, —O—, —S— or —PR¹—;
M is B, Si or Sn;
L is C₁-C₆ alkyl, -Aryl or —F
n is 1 or 2; o is an integer between 2 and 10;

Due to practical reasons, a more preferred embodiment of the invention comprise compounds where the carrier is selected from the group consisting of: 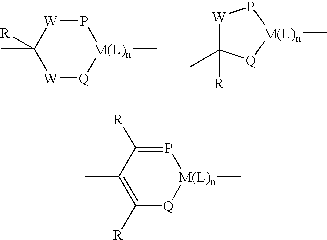
wherein
W is —CR¹R¹′—, —C(═O)—, —C(═S)—, —C(═NR²)— or —NR¹—;
P and Q are independently chosen from —CR¹R¹′—, —NR¹—, —O—, —S— or —PR¹—;
M is B, Si or Sn;
L is C₁-C₆ alkyl, -Aryl or —F;
n is 1 or 2;

4. A compound according to claim 1 wherein the Spacer is a valence bond, C₁-C₆ alkylene-A-, C₂-C₆ alkenylene-A-, C₂-C₆ alkynylene-A-, or 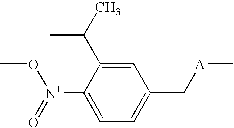
said spacer optionally being connected through A to a linker selected from 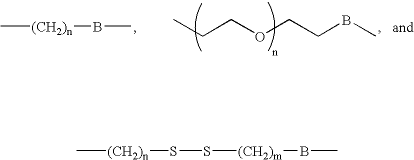
where A is a valence bodn, —C(O)N—, —N—, —O—, —S—, or —C(O)—O—; B is a valence bond, —O—, —S—, —N— or —C(O)N— and connects to S—C-connecting group; R⁸ is selected independently from H, C₁-C₆ alkyl, C₃-C₇ cycloalkyl, aryl or C₁-C₆ alkylene-aryl and n and m independently are integers ranging from 1 to 10,

5. A compound according to claim 1 wherein the S—C-connecting group is a valence bond, —NH—C(═O)—, —NH—C(═O)—C₁-C₆ alkylene-, —S—S—, —S—S—C₁-C₆ alkylene-, —C(═O)—NH—, —C(═O)—NH—(C₁-C₆ alkylene)-, 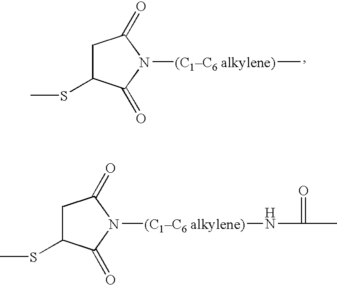
—NH—C(═O)-Arylene-C( )₂—NH—C(═O)—.

In another more preferred embodiment of the invention, the carrier is -Aryl-B(L)₂-where L is independently chosen from aryl or —F.

The S—C-connecting group provide a means for connecting the Spacer and the Carrier. As such it is primarily of synthetic convenience and does not influence the function of a building block.

The spacer serves to distance the functional entity to be transferred from the bulky complementing element. Thus, when present, the identity of the spacer is not crucial for the function of the building block. It may be desired to have a spacer which can be cleaved by light. In this occasion, the spacer is provided with e.g. the group

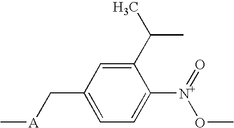

In the event an increased hydrophilicity is desired the spacer may be provided with a polyethylene glycol part of the general formula:

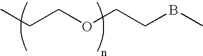

In a preferred embodiment, the complementing element serves the function of recognising a coding element. The recognition implies that the two parts are capable of interacting in order to assemble a complementing element—coding element complex. In the biotechnological field a variety of interacting molecular parts are known which can be used according to the invention. Examples include, but are not restricted to protein-protein interactions, protein-polysaccharide interactions, RNA-protein interactions, DNA-DNA interactions, DNA-RNA interactions, RNA-RNA interactions, biotin-streptavidin interactions, enzyme-ligand interactions, antibody-ligand interaction, protein-ligand interaction, ect.

The interaction between the complementing element and coding element may result in a strong or a weak bonding. If a covalent bond is formed between the parties of the affinity pair the binding between the parts can be regarded as strong, whereas the establishment of hydrogen bondings, interactions between hydrophobic domains, and metal chelation in general results in weaker bonding. In general relatively weak bonding is preferred. In a preferred aspect of the invention, the complementing element is capable of reversible interacting with the coding element so as to provide for an attachment or detachment of the parts in accordance with the changing conditions of the media.

In a preferred aspect of the invention, the interaction is based on nucleotides, i.e. the complementing element is a nucleic acid. Preferably, the complementing element is a sequence of nucleotides and the coding element is a sequence of nucleotides capable of hybridising to the complementing element. The sequence of nucleotides carries a series of nucleobases on a backbone. The nucleobases may be any chemical entity able to be specifically recognized by a complementing entity. The nucleobases are usually selected from the natural nucleobases (adenine, guanine, uracil, thymine, and cytosine) but also the other nucleobases obeying the Watson-Crick hydrogen-bonding rules may be used, such as the synthetic nucleobases disclosed in U.S. Pat. No. 6,037,120. Examples of natural and non-natural nucleobases able to perform a specific pairing are shown in FIG. 2. The backbone of the sequence of nucleotides may be any backbone able to aggregate the nucleobases is a sequence. Examples of backbones are shown in FIG. 4. In some aspects of the invention the addition of non-specific nucleobases to the complementing element is advantegeous, FIG. 3.

The coding element can be an oligonucleotide having nucleobases which complements and is specifically recognised by the complementing element, i.e. in the event the complementing element contains cytosine, the coding element part contains guanine and visa versa, and in the event the complementing element contains thymine or uracil the coding element contains adenine.

The complementing element may be a single nucleobase. In the generation of a library, this will allow for the incorporation of four different functional entities into the template-directed molecule. However, to obtain a higher diversity a complementing element preferably comprises at least two and more preferred at least three nucleotides. Theoretically, this will provide for 4² and 4³, respectively, different functional entities uniquely identified by the complementing element. The complementing element will usually not comprise more than 100 nucleotides. It is preferred to have complementing elements with a sequence of 3 to 30 nucleotides.

The building blocks of the present invention can be used in a method for transferring a functional entity to a recipient reactive group, said method comprising the steps of
providing one or more building blocks as described above and
contacting the one or more building blocks with a corresponding encoding element associated with a recipient reactive group under conditions which allow for a recognition between the one or more complementing elements and the encoding elements, said contacting being performed prior to, simultaneously with, or subsequent to a transfer of the functional entity to the recipient reactive group.

The encoding element may comprise one, two, three or more codons, i.e. sequences that may be specifically recognised by a complementing element. Each of the codons may be separated by a suitable spacer group. Preferably, all or at least a majority of the codons of the template are arranged in sequence and each of the codons are separated from a neighbouring codon by a spacer group. Generally, it is preferred to have more than two codons on the template to allow for the synthesis of more complex encoded molecules. In a preferred aspect of the invention the number of codons of the encoding element is 2 to 100. Still more preferred are encoding elements comprising 3 to 10 codons. In another aspect, a codon comprises 1 to 50 nucleotides and the complementing element comprises a sequence of nucleotides complementary to one or more of the encoding sequences.

The recipient reactive group may be associated with the encoding element in any appropriate way. Thus, the reactive group may be associated covalently or noncovalently to the encoding element. In one embodiment the recipient reactive group is linked covalently to the encoding element through a suitable linker which may be separately cleavable to release the reaction product. In another embodiment, the reactive group is coupled to a complementing element, which is capable of recognising a sequence of nucleotides on the encoding element, whereby the recipient reactive group becomes attached to the encoding element by hybridisation. Also, the recipient reactive group may be part of a chemical scaffold, i.e. a chemical entity having one or more reactive groups available for receiving a functional entity from a building block.

The recipient reactive group may be any group able to participate in cleaving the bond between the carrier and the functional entity precursor to release the functional entity precursor. Usually, the reactive group is an electronegative atom such as —OR, —F, —Cl, —Br or —I where R is a substituted sulfonyl group (ie.—OR comprises -OMs, -OTf and -OTos) activated by a transition metal such as Pd, Pt, Ni, Cu, Rh or Ru. Typically, the reactive group is attached to an aromatic- or heteroaromatic ring (Scheme 1) or a C—C double bond (Scheme 2). Scheme 3 shows an alkyl or alkenyl Functional Entity replacing a reactive recipient group attached to an aryl.

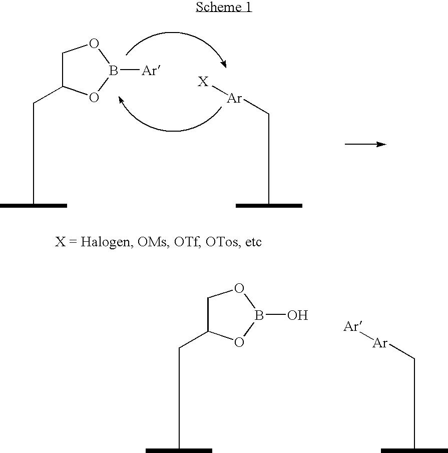

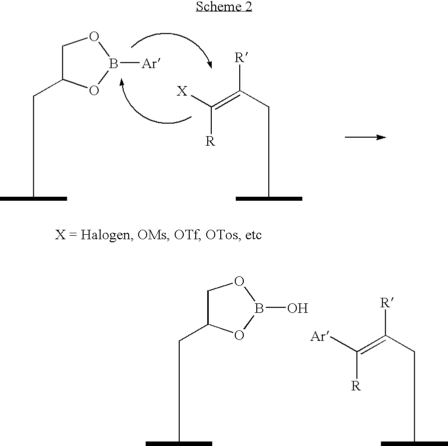

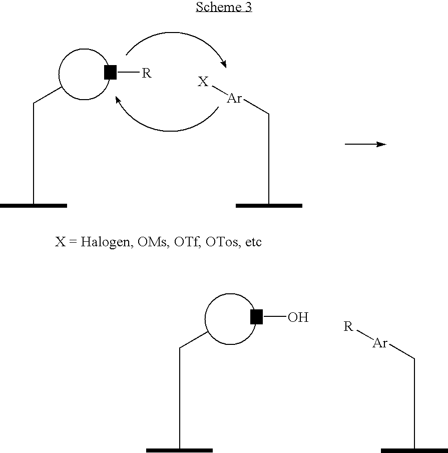

Also aldehydes or imines may serve as recipient reactive group optionally in the presence of a catalyst.

According to a preferred aspect of the invention the building blocks are used for the formation of a library of compounds. The complementing element of the building block is used to identify the functional entity. Due to the enhanced proximity between reactive groups when the complementing entity and the encoding element are contacted, the functional entity together with the identity programmed in the complementing element is transferred to the encoding element associated with recipient reactive group. Thus, it is preferred that the sequence of the complementing element is unique in the sense that the same sequence is not used for another functional entity. The unique identification of the functional entity enable the possibility of decoding the encoding element in order to determine the synthetic history of the molecule formed. In the event two or more functional entities have been transferred to a scaffold, not only the identity of the transferred functional entities can be determined. Also the sequence of reaction and the type of reaction involved can be determined by decoding the encoding element. Thus, according to a preferred embodiment of the invention, each different member of a library comprises a complementing element having a unique sequence of nucleotides, which identifies the functional entity.

### BRIEF DESCRIPTION OF THE DRAWINGS

FIG. 1. Two setups for Functional Entity Transfer

FIG. 2. Examples of specific base pairing

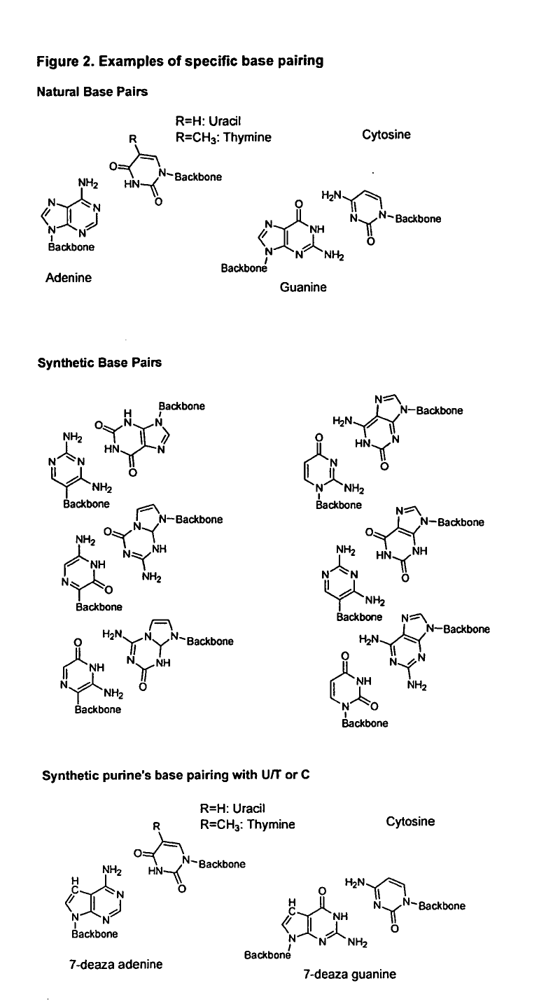

FIG. 3. Example of non-specific base-pairing

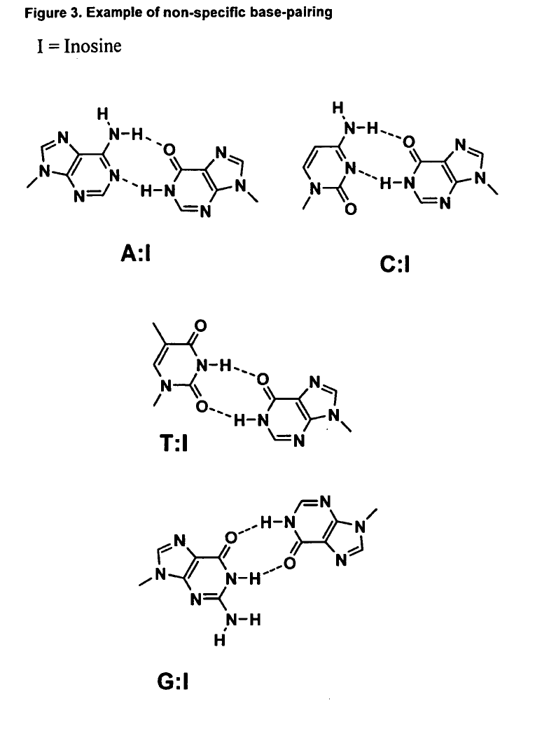

FIG. 4. Backbone examples

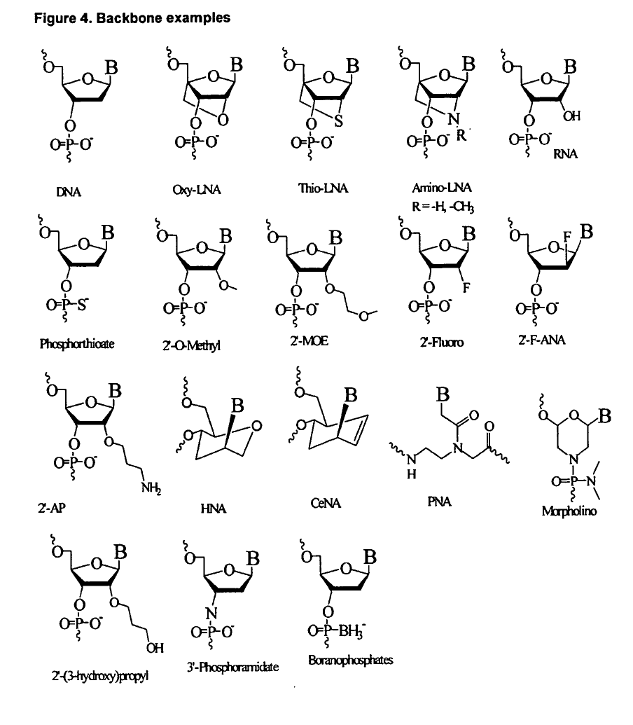

### DETAILED DESCRIPTION OF THE INVENTION

A building block of the present invention is characterized by its ability to transfer its functional entity to a recipient reactive group. This is done by forming a new covalent bond between the recipient reactive group and cleaving the bond between the carrier moiety and the functional entity of the building block.

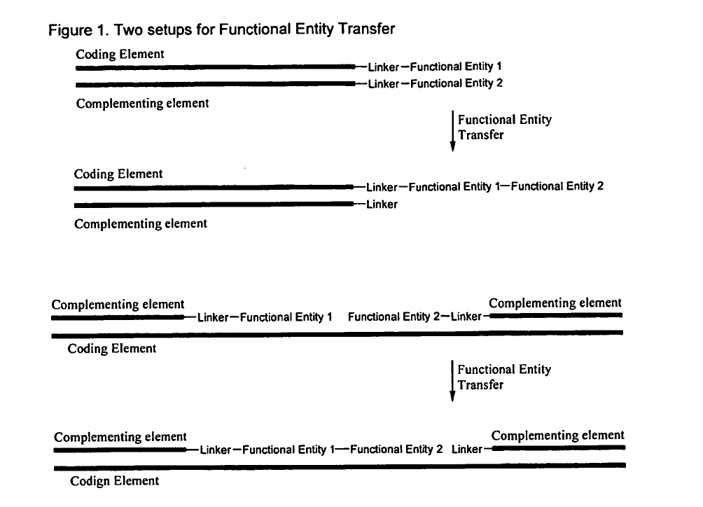

Two setups for generalized functional entity transfer from a building block are depicted in FIG. 1. In the first example, one complementing element of a building block recognizes a coding element carrying another functional entity, hence bringing the functional entities in close proximity. This results in a reaction between functional entity 1 and 2 forming a covalent bond between these concurrent with the cleavage of the bond between functional entity 2 and its linker. In the second example, a coding element brings together two building blocks resulting in functional entity transfer from one building block to the other.

### EXPERIMENTAL SECTION

Assembly of Building Blocks

The Carrier-Functional Entity ensemble may be bound to the Spacer by several different reactions as illustrated below.
Formation of an Amide Bond between a Carboxylic Acid of the Carrier and an Amine Group of a Spacer 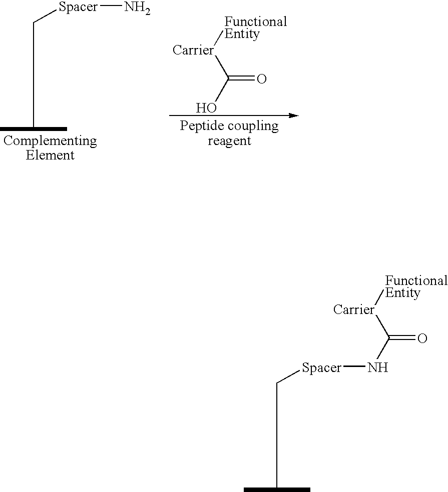
General Procedure 1: Preparation of Neutral Boronic Ester Derivatives (I):

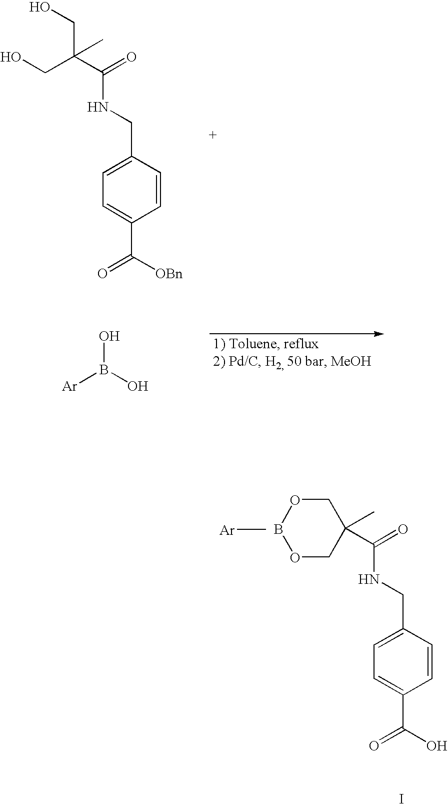

4-[(3-Hydroxy-2-hydroxymethyl-2-methyl-propionylamino)-methyl]-benzoic acid benzyl ester (0.59 mmol, 210 mg) and aryl boronic acid (0.60 mmol) is mixed in toluene (15 mL) and stirred 16 h at 70° C. The product is obtained by evaporation of the solvent under reduced pressure.

The aryl boronic acid derivate (0.12 mmol) is dissolved in methanol and transferred to an autoclave. A catalytic amount of palladium on activated carbon (5 wt. %) is added to the solution under an argon atmosphere. The argon is exchanged with hydrogen and the reaction is performed at room temperature for 24 hours under a pressure of 50 bars affording I upon filtration and removal of the solvent.

### Example 1

### General Procedure (1)

### 4-({[2-(4-Fluoro-phenyl)-5-methyl-[1,3,2]dioxaborinane-5-carbonyl]-amino}-methyl)benzoic acid

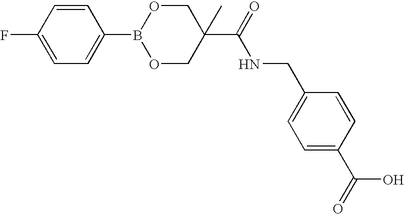

Yield 90% (0.11 mmol, 40 mg). ¹H-NMR (DMSO-d₆): 8.59 (t, 1H); 7.70-7.11 (m, 8H); 4.44 (d, 2H); 4.36 (d, 2H); 3.96 (d, 2H); 1.13 (s, 3H)
Synthesis of the Boronic Ester Ligand (II):

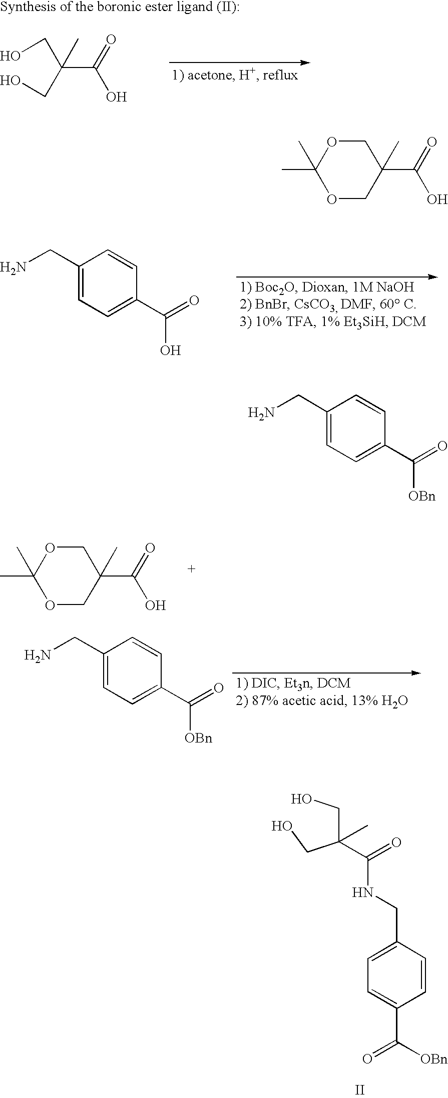

Isopropylidene-2,2-bis(hydroxymethyl)propionic acid:

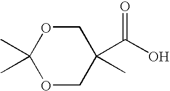

2,2-Bis(hydroxymethyl)propionic acid (0.12 mol, 15.9 g) was refluxed in acetone (250 mL) with molecular sieves and conc. sulphuric acid (0.5 mL) for 10 hours. The reaction mixture was then neutralised with NaHCO₃ (1 M aq.), stirred with activated charcoal and filtered. The product was collected as a white crystalline upon concetration of the solvent.

Yield 50% (10.5 g): ¹H-NMR (DMSO-d₆): 1.07 (s, 3H, —CH₃); 1.26 (s, 3H, —CH₃); 1.34 (s, 3H, —CH₃); 3.53 and 3.57 (d, 2H, —CH₂—); 3.99 and 4.02 (d, 2H, —CH₂—).

4-(Boc-amino-methyl)-benzoic acid:

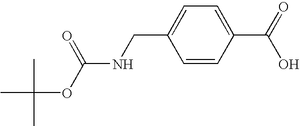

4-Methylaminobenzoic acid was dissolved in dioxane (10 mL) and NaOH (22 mL, 1M solution) and cooled to 0° C. Ditertbutyl dicarbonate (10 mmol, 2.18 g) and NaOH (8 mL, 2M solution) was added, and the reaction mixture was left over night at room temperature. Half of the solvent was removed under reduced pressure and ethylacetate added (25 mL). The reaction mixture was then neutralised by adding HCl (2 M solution) to pH=4, and extracted with ethyl acetate (3×75 mL). The organic phase was dried, and evaporated to dryness, and the product was obtained as a white crystalline solid.

Yield: 65% (6.0 mmol, 1.51 g): ¹H-NMR (DMSO-d₆): 12.84 (s, 1H); 7.89 (d, 2H); 7.46 (t, 1H); 7.34 (d, 2H); 4.19 (d, 2H); 1.40 (s, 9H).

4-[(Boc-amino)-methyl]-benzoic acid benzyl ester:

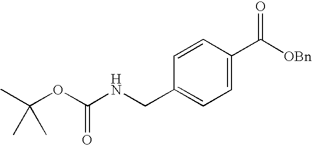

4-[(Boc-amino)-methyl]-benzoic acid (5.89 mmol, 1.48 g) in anhydrous DMF (20 mL) was added Cs₂CO₃ (2.95 mmol, 0.96 g) and stirred for 1 h at room temperature. Benzyl bromide (8.2 mmol, 1.0 mL) was added, and the reaction stirred for 9 hours. The solvent was removed under reduced pressure, and the crude was suspended in water (100 mL) and extracted with diethyl ether (3×100 mL). The organic phase was then dried, evaporated to dryness and the obtained product was purified using dry column vacuum chromatography.

Yield=81% (4.79 mmol, 1.56 g): ¹H-NMR (DMSO-d₆): 7.95 (d, 2H); 7.48-7.37 (m, 7H); 5.35 (s, 2H); 4.20 (d, 2H); 1.39 (s, 9H).

4-Methylamino benzoic acid benzyl ester:

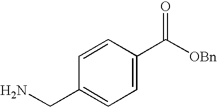

N-Boc-4-methylamino benzoic benzyl ester (4.79 mmol, 1.55 g) was dissolved in DCM (25 mL) with TFA (10% v/v) and triethylsilane (1% v/v) and stirred for 30 minutes. The solvent was removed under reduced pressure and the product purified using dry column vacuum chromatography.

Yield=47% (2.28 mmol, 550 mg): ¹H-NMR (DMSO-d₆): 8.69 (s, 2H); 8.03 (d, 2H); 7.62 (d, 2H); 7.50-7.36 (m, 5H); 5.37 (s, 2H); 4.14 (s, 2H).

4-{[(2,2,5-Trimethyl-[1,3]dioxane-5-carbonyl)-amino]-methyl}benzoic acid benzyl ester

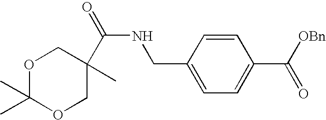

Isopropylidene-2,2-bis(hydroxymethyl)propionic acid (4.10 mmol, 714 mg) and 4-methylamino benzyloxy benzoic acid (4.14 mmol, 1.0 g) in DCM (20 mL) was cooled to 0° C. and diisopropyl carbodiimide (5.5 mmol, 0.7 mL) was added. The reaction mixture was left over night at room temperature, and the solvent was removed under reduced pressure. The crude was dissolved in toluene and filtered. The filtrate was purified using Dry Column Vacuum Chromatography.

Yield=29% (478 mg): ¹H-NMR (DMSO-d₆): 8.25 (t, 1H); 7.93 (d, 2H); 7.47-7.35 (m, 9H); 5.34 (s, 2H); 4.39 (d, 2H); 4.04 (d, 2H); 3.65 (d, 2H); 1.37 (s, 3H); 1.29 (s, 3H); 1.05 (s, 3H);

4-[(3-Hydroxy-2-hydroxymethyl-2-methyl-propionylamino)-methyl]-benzoic acid benzyl ester

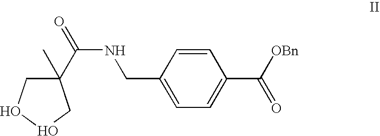

4-{[(2,2,5-Trimethyl-[1,3]dioxane-5-carbonyl)-amino]-methyl}-benzoic acid benzyl ester (1.2 mmol, 478 mg) was dissolved in acetic acid (11.5 mL, 87% v/v) and stirred at 40° C. for 3 hours. The product II was obtained by evaporation of the reaction mixture under reduced pressure and co evaporation from anhydrous toluene (3×20 mL).

Yield=90%: ¹H-NMR (DMSO-d₆): 8.07 (t, 1H); 7.92 (d, 2H); 7.48-7.12 (m, 7H); 5.34 (s, 2H); 4.72 (bs, 2H); 4.37 (d, 2H); 3.46 (m, 4H); 1.04 (s, 3H).

### Example 2

### General procedure (1)

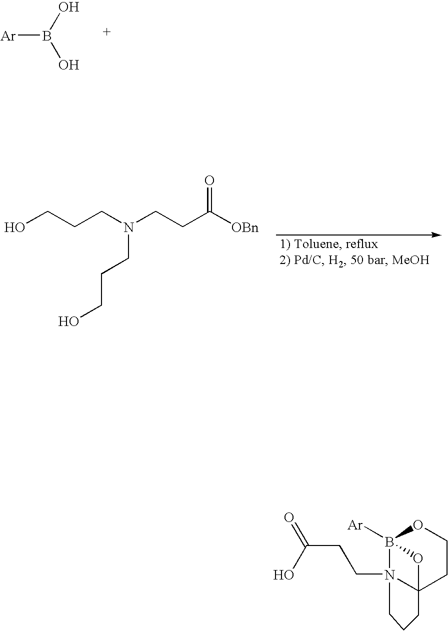 Synthesis of the Boronic Ester Ligand (III):

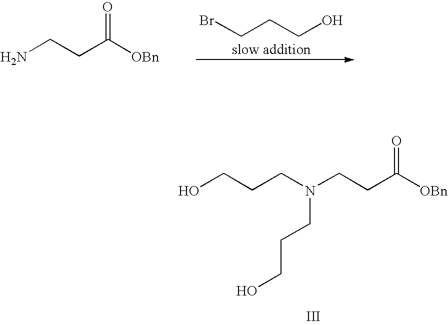

3-[Bis-(3-hydroxy-propyl)-amino]-propionic acid benzyl III ester is synthesised according to literature procedures from the corresponding 3-amino-propionic acid benzyl ester (Goldschmidt; Veer; RTCPA3; Recl. Trav. Chim. Pays-Bas; 1948, 67, 489.)
General Procedure 2: Synthesis of Flouroborate Cesium Salt Derivatives:

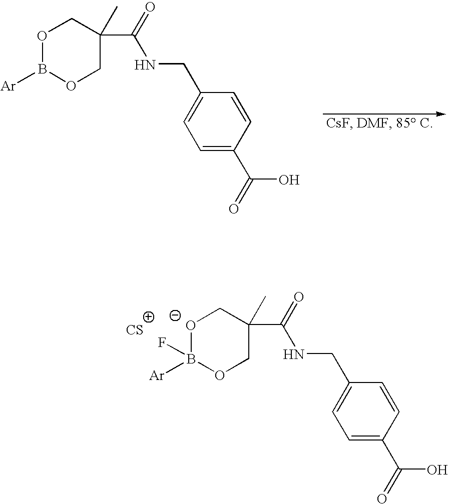

Caesium fluoride (18 mg, 0.12 mmol) is added to a stirred solution of the aryl boronic ester derivate (0.12 mmol) in DMF (4 mL) at 85° C. The mixture is stirred for 3 hours. The product precipitates from solution during evaporation of the solvent under reduced pressure. Upon filtration the product was filtered and washed with diethyl ether.

### Example 3

### General procedure (2)

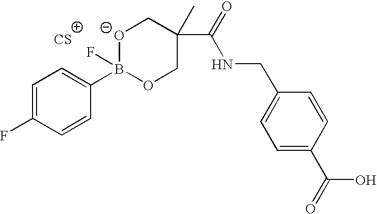

Yield=40% (0.048 mmol, 25 mg) ¹H-NMR (DMSO-d₆): 8.06 (t, 1H); 7.88-7.14 (m, 8H); 4.73 (t, 2H); 4.45 (d, 1H); 4.36 (d, 2H); 3.97 (d, 1H); 1.04 (s, 3H).

¹⁹F-NMR (DMSO-d₆): −74.75, −109.76, −118.89, −139.00, −148.28.
General Procedure 3: Synthesis of Fluoroborate Potassium Salt Derivatives:

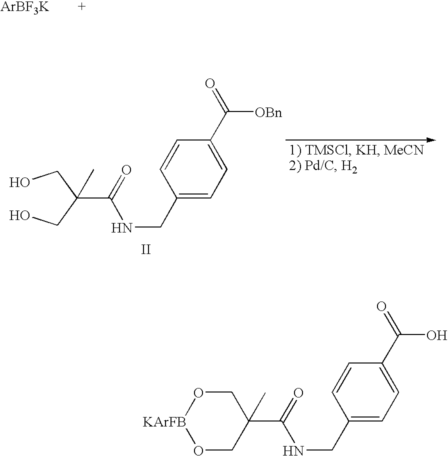

Potassium hydride (80 mg, 2.0 mmol) is added to a stirred solution of 4-[(3-hydroxy-2-hydroxymethyl-2-methyl-propionylamino)-methyl]-benzoic acid benzyl ester II (357 mg, 1.0 mmol) in anhydrous acetonitrile (10 mL) at room temperature. Potassium aryltrifluoroborate (1.0 mmol) was added to the reaction mixture, followed by chlorotrimethylsilane (231 μL, 2.0 mmol). The mixture is stirred for 2 hour at room temperature and then diluted with ethyl acetate (40 mL), washed with distilled water (2×40 mL) and dried over sodium sulphate (anhydrous). Removal of solvent yields a crude product which is purified by dissolving in hot acetone and precipitating with petroleum ether.

The fluoroborate potassium salt derivate (0.5 mmol) is dissolved in methanol and transferred to an autoclave. A catalytic amount of palladium on activated carbon (5 wt. %) is added to the solution under an argon atmosphere. The argon was exchanged with hydrogen and the reaction is performed at room temperature for 24 hours under a pressure of 50 bars affording the desired product upon filtration and removal of the solvent.
General Procedure 4: Synthesis of Fluoroborate Potassium Salt Derivatives:

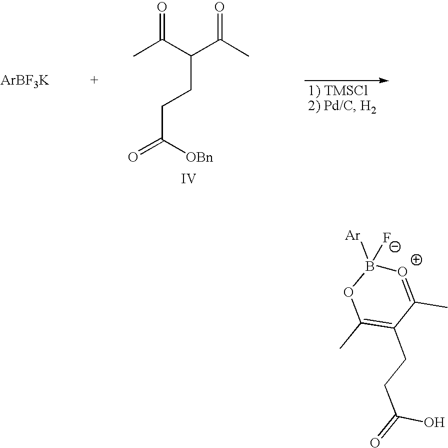

Chlorotrimethyl silane (231 μL, 2.0 mmol) is added to a stirred solution of potassium aryltrifluoroborate (IV) (1.0 mmol) and 4-acetyl-5-oxo-hexanoic acid benzyl ester (262 mg, 1.0 mmol) in anhydrous acetonitrile (10 mL) at room temperature under an atmosphere of nitrogen. The mixture is stirred for 1 hour at room temperature and then diluted with ethyl acetate (40 mL), washed with distilled water (2×40 mL) and dried over sodium sulphate. Removal of solvent gives a crude product, which was subjected to plug filtration on silica gel (dichloromethane/heptane 50:50). The fluoroborate derivate (0.5 mmol) is dissolved in methanol and transferred to an autoclave. A catalytic amount of palladium on activated carbon (5 wt. %) is added to the solution under an argon atmosphere. The argon is exchanged with hydrogen and the reaction is performed at room temperature for 24 hours under a pressure of 50 bars affording the desired product upon filtration and removal of the solvent.

### Example 4

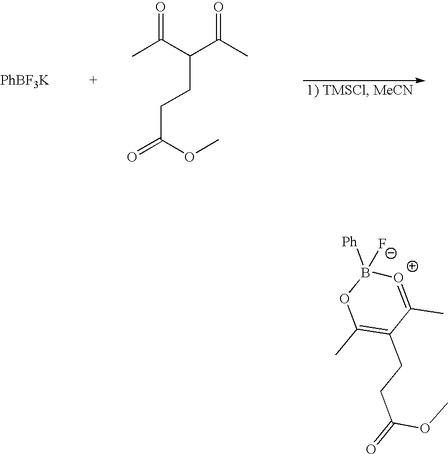

To a stirred solution of potassium phenyltrifluoroborate (204 mg, 1.11 mmol) and methyl 4-acetyl-5-oxo-hexanoate (194 μL, 1.11 mmol) in anhydrous acetonitrile (5 mL) was added chlorotrimethyl silane (257 μL, 2.22 mmol) at room temperature under an atmosphere of nitrogen. The mixture was stirred overnight at room temperature and then diluted with ethyl acetate (20 mL), washed with distilled water (2×20 mL) and dried over sodium sulphate. Removal of solvent gave an oil, which was subjected to plug filtration on silica gel (dichloromethane/heptane 50:50) to give.

Yield=37%: ¹H-NMR (CDCl₃): 7.55 (dd, 2H); 7.38-7.30 (m, 3H); 3.72 (s, 3H); 2.76-2.71 (m, 2H); 2.52-2.47 (m, 2H); 2.40 (s, 6H); ¹⁹F-NMR (CDCl₃): −143.7 (s) (without internal standard).
General Procedure 5: Preparation of Difluoroborate Potassium Salt Derivatives (V):

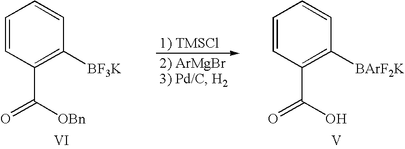

To a stirred solution of potassium aryltrifluoroborate (VI) (1.0 mmol) in anhydrous THF is added TMSCl (1.0 mmol) at room temperature under an atmosphere of nitrogen. After 1 h, the mixture is cooled to −10° C. and aryl magnesiumbromide (1.0 mmol) is added. The mixture is stirred for 1 hour at room temperature and then diluted with ethyl acetate (40 mL), washed with distilled water (2×40 mL) and dried over sodium sulphate (anhydrous). Removal of solvent gives a crude product which is purified by dissolving in hot acetone and precipitating with petroleum ether. The difluoroborate potassium salt derivate (0.5 mmol) is dissolved in methanol and transferred to an autoclave. A catalytic amount of palladium on activated carbon (5 wt. %) is added to the solution under an argon atmosphere. The argon is exchanged with hydrogen and the reaction is performed at room temperature for 24 hours under a pressure of 50 bars affording the desired product upon filtration and removal of the solvent.
Synthesis of Borate (VI):

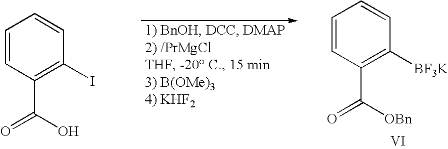

The potassium aryltrifluoroborate (VI) was synthesised in according to literature procedures from the corresponding 2-iodo-benzoic acid. (Molander, G. A.; Biolatto, B. Org. Lett. 2002, 4, 1867., Molander, G. A.; Katona, B. W.; Machrouhi, F. J. Org. Chem. 2002, 67, 8416., Molander, G. A.; Bernardi, C. J. Org. Chem. 2002, 67, 8224.)

Yield=35%: ¹H-NMR (DMSO-d₆): 7.48-7.44 (m, 3H); 7.35-7.27 (m, 3H); 7.20 (d, 2H); 7.12-7.09 (m, 1H); 5.16 (s, 1H); ¹⁹F-NMR (DMSO-d₆): −137.20 (m) (without internal standard).

### Example 5

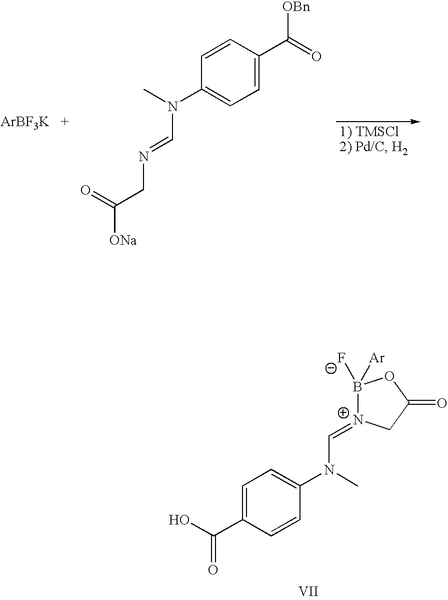

The oxazaborolidinone VII is synthesised according to literature procedures for the corresponding sodium salt of 4-[(N-carboxymethyl-formimidoyl)-methyl-amino]-benzoic acid benzyl ester VII and potassium aryltrifluoroborate.(Vedejs, E.; Chapman, R. W.; Fields, S. C.; Lin, S.; Schrimpf, M. R. J. Org. Chem. 1995, 60, p 3020.)
Synthesis of Ligands for Oxazaborolidinones:

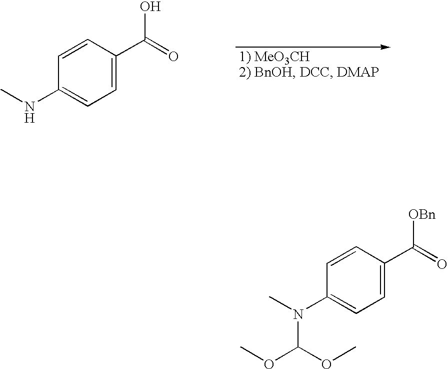

The 4-(dimethoxymethyl methyl-amino)-benzoic acid benzyl ester is synthesised according to literature procedures from the corresponding 4-methylamino-benzoic acid.(Scheeren, J. W.; Nivard, R. J. F.; RTCPA3; Recl. Trav. Chim. Pays-Bas; 1969, 88, 3, 289.) The acetal derivate from the first step (315 mg, 1.0 mmol) is dissolved in dichloromethane (10 mL) followed by addition of benzyl alcohol (119 mg, 1.1 mmol), DCC (227 mg, 1.1 mmol) and DMAP (12.2 mg, 0.1 mmol). The reaction mixture is stirred overnight at 25° C. The solvent is evaporated under reduced pressure and the crude purified on column chromatography using silica gel.

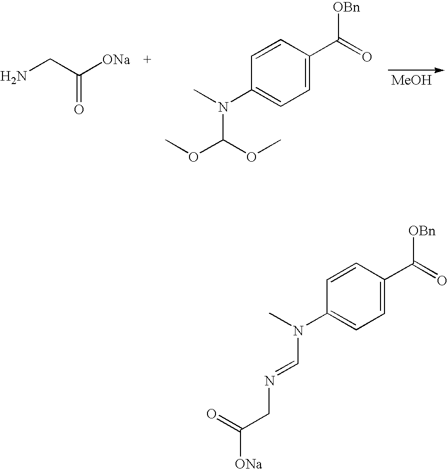

The sodium salt of 4-[(N-carboxymethyl-formimidoyl)-methyl-amino]-benzoic acid benzyl ester is synthesised in according to literature procedures from the corresponding 4-(dimethoxymethyl-methyl-amino)-benzoic acid benzyl ester and the sodium salt of glycine. (Vedejs, E.; Chapman, R. W.; Fields, S. C.; Lin, S.; Schrimpf, M. R. J. Org. Chem. 1995, 60, p 3020.)
General Procedure 6: Preparation of Building Blocks by Loading a Carrier-Functional Entity Ensemble onto a Nucleotide Derivative Comprising an Amino Group:

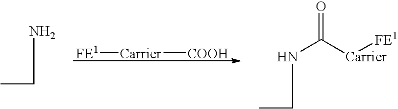

15 μL of a 150 mM building block solution of FE¹-Carrier-COOH is mixed with 15 μL of a 150 mM solution of EDC and 15 μL of a 150 mM solution of N-hydroxysuccinimide (NHS) using solvents like DMF, DMSO, water, acetonitril, THF, DCM, methanol, ethanol or a mixture thereof. The mixture is left for 15 min at 25° C. 45 μL of an aminooligo (10 nmol) in 100 mM buffer at a pH between 5 and 10, preferably 6.0-7.5 is added and the reaction mixture is left for 2 hours at 25° C. Excess building block and organic by-products were removed by extraction with EtOAc (400 μL). Remaining EtOAc is evaporated in vacuo using a speedvac. The building block is purified following elution through a BioRad micro-spin chromatography column, and analyzed by electron spray mass spectrometry (ES-MS).

Use of Building Blocks

General Procedure 7: C—C Coupling Between Oligonucleotide Derivatives Containing an Recipient Reactive Group and a Building Block According to the Invention:

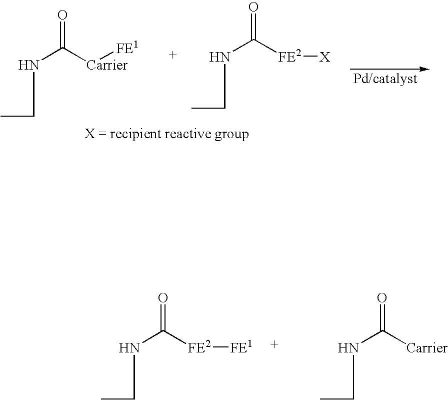

An oligonucleotide building block carrying functional entity FE¹ is combined at 2 μM final concentration with one equivalent of a complementary building block displaying an organo-halide or organo-triflate. Reaction proceeds at temperatures between 0° C. and 100° C. preferably between 15° C.-50° C. for 148 hours, preferably 10-20 hours in DMF, DMSO, water, acetonitril, THF, DCM, methanol, ethanol or a mixture thereof, pH buffered to 4-10, preferably 6-8 in the presence of a Pd catalyst. Organic by-products are removed by extraction with EtOAc, followed by evaporation of residual organic solvent for 10 min in vacuo. Pd catalyst is removed and oligonucleotides are isolated by eluting sample through a BioRad micro-spin chromatography column. Coupling efficiency is quantified by ES-MS analysis.

### Example 6

### An Illustration of the Entire Process from Building Block Synthesis to Functional Entity Transfer

Nucleophilic monomer building blocks capable of transferring an aryl, hetaryl or vinyl functionality may be prepared from organic building blocks type (3). This is available by estrification of a boronic acid by a diol e.g. (1), followed by transformation into the NHS-ester derivative. The NHS-ester derivative may then be coupled to an oligonucleotide to generate monomer building block type (5). Alternatively, the carboxylic acid (2) may be used in general procedure 6.

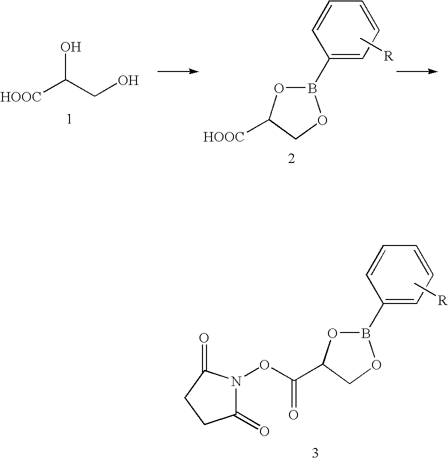

Likewise, building block 4 may be prepared via an NHS-ester or by general procedure 6:

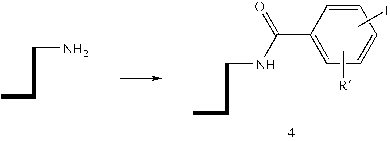

The transtion metal catalyzed cross coupling is conducted as follows:

A premix of 1.4 mM Na₂PdCl₄ and 2.8 mM P(p-SO₃C₆H₄)₃ in water left for 15 min was added to a mixture of the template oligonucleotide (1 nmol) and monomer building block (4) and (5) (both 1 nmol) in 0.5 M NaOAc buffer at pH=5 and 75 mM NaCl (final [Pd]=0.3 mM). The mixture is then left o/n at 35-65° C. preferably 58° C., to yield template bound (6).

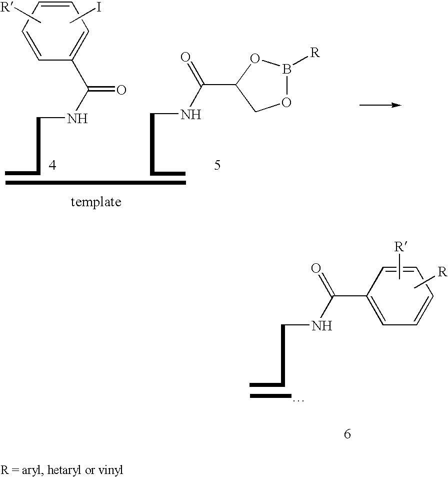

R=aryl, hetaryl or vinyl

Abbreviations

| | |
|---|---|
| DCC | N,N′-Dicyclohexylcarbodiimide |
| DhbtOH | 3,4-dihydro-3-hydroxy-4-oxo-1,2,3-benzotriazine |
| DIC | Diisopropylcarbodiimide |
| DIEA | Diethylisopropylamin |
| DMAP | 4-Dimethylaminopyridine |
| DNA | Deoxyribosenucleic Acid |
| EDC | 1-Ethyl-3-(3′-dimethylaminopropyl)carbodiimide.HCl |
| HATU | 2-(1H-7-Azabenzotriazole-1-yl)-1,1,3,3-tetramethyluronium |
| | hexafluorophosphate |
| HBTU | 2-(1H-Benzotriazole-1-yl)-1,1,3,3-tetramethyluronium |
| | hexafluorophosphate |
| HOAt | N-Hydroxy-7-azabenzotriazole |
| HOBt | N-Hydroxybenzotriazole |
| LNA | Locked Nucleic Acid |
| NHS | N-hydroxysuccinimid |
| OTf | Trifluoromethylsulfonate |
| OTs | Toluenesulfonate |
| PNA | Peptide Nucleic Acid |
| PyBoP | Benzotriazole-1-yl-oxy-tris-pyrrolidino-phosphonium |
| | hexafluorophosphate |
| PyBroP | Bromo-tris-pyrrolidino-phosphonium hexafluorophosphate |
| RNA | Ribonucleic acid |
| TBTU | 2-(1H-Benzotriazole-1-yl)-1,1,3,3-tetramethyluronium |
| | tetrafluoroborate |
| TEA | Triethylamine |
| RP-HPLC | Reverse Phase High Performance Liquid Chromatography |
| TBDMS-CI | Tert-Butyldimethylsilylchloride |
| 5-Iodo-dU | 5-iodo-deoxyriboseuracil |
| TLC | Thin layer chromatography |
| (Boc)₂O | Boc anhydride, di-tert-butyl dicarbonate |
| TBAF | Tetrabutylammonium fluoride |
| SPDP | Succinimidyl-propyl-2-dithiopyridyl |

1. A building block of the general formula
Complementing Element-Linker-Carrier-Functional entity precursor
capable of transferring a functional entity to a recipient reactive group, wherein
Complementing Element is a group identifying the functional entity,
Linker is a chemical moiety comprising a spacer and a S—C-connecting group, wherein the spacer is a valence bond or a group distancing the functional entity precursor to be transferred from the complementing element and the S—C-connecting group connects the spacer with the Carrier,
Carrier comprises an aromatic-, a saturated- or a partially saturated heterocyclic ring system, said ring system being mono-, di- or tricyclic and substituted with 0-3 R¹ and containing a ring-member M belonging to the group consisting of B, Si, Sn and Zn, whereas M carries the functional entity precursor and 0-2 ligands L selected independently from the group consisting of —F, -aryl, -heteroaryl, or
Carrier is —Ar-M(L)ₚ—, —Ar-(C₁-C₆ alkylene)-M(L)ₚ— or —Ar—X—(C₁-C₆ alkylene)-M(L)ₚ— where Ar is aryl or heteroaryl substituted with 0-3 R¹, M² is B, Sn or Si, X is O, S, or R² and L is independently chosen from —F, -aryl, -heteroaryl or C₁-C₆ alkyl; R¹ and R^{1′} are independently selected from —H, —O R², —N R²₂, -Halogen, —NO₂, —CN, —C(Halogen)₃, —C(O)R², —C(O)NHR², C(O)NR²₂, —NC(O)R², —S(O)₂NHR², —S(O)₂NR²₂, —S(O)₂R², —P(O)₂—R², —P(O)—R², —S(O)—R², P(O)—OR², —S(O)—OR², N⁺R²₃, wherein p is an integer of 0 to 3 and R² is H, C₁-C₆ alkyl, C₂-C₆ alkenyl, C₂-C₆ alkynyl, or aryl,
Functional entity precursor is H or selected among the group consisting of a C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R³, 0-3 R⁴ and 0-3 R⁷ or C₁-C₃ alkylene-NR³₂, C₁-C₃ alkylene-NR³C(O)R⁶, C₁-C₃ alkylene-NR³C(O)OR⁶, C₁-C₂ alkylene-O—N R³₂, C₁-C₂ alkylene-O—NR³C(O)R⁶, C₁-C₂ alkylene-O—N R³C(O)OR⁶ substituted with 0-3 R⁷.
where R³ is H or selected independently among the group consisting of C₁-C₆ alkyl, C₂-C₆ alkenyl, C₂-C₆ alkynyl, C₃-C₇ cycloalkyl, C₃-C₇ cycloheteroalkyl, aryl, heteroaryl, said group being substituted with 0-3 R⁴ and 0-3 R⁷
R⁴ is selected independently from —N₃, —CNO, —C(NOH)NH₂, —NHOH, —NHNH, —C(O), —P(O)(O)₂ or the group consisting of C₂-C₆ alkenyl, C₂-C₆ alkynyl, C₄-C₈ alkadienyl said group being substituted with 0-2 R⁵,
R⁵ is independently selected from —NO₂, —C(O)O, —C(O), —CN, —OSi₃, —O and —N₂, and
R⁶ is H, C₁-C₆ alkyl, C₂-C₆ alkenyl, C₂-C₆ alkynyl, C₃-C₇ cycloalkyl, aryl or C₁-C₆ alkylene-aryl substituted with 0-3 substituents independently selected from —F, —Cl, —NO₂, —R², —OR², —SiR²₃, and
R⁷ is ═O, —F, —Cl, —Br, —I, —CN, —NO₂, -O, —N₂, —N—C(O)R⁶, —N—C(O)OR⁶, —S, —(O), —S(O)₂, —COO, —C(O)N₂, or —S(O)₂N₂.

2. A compound according to claim 1 wherein, the carrier is selected from the group consisting of: 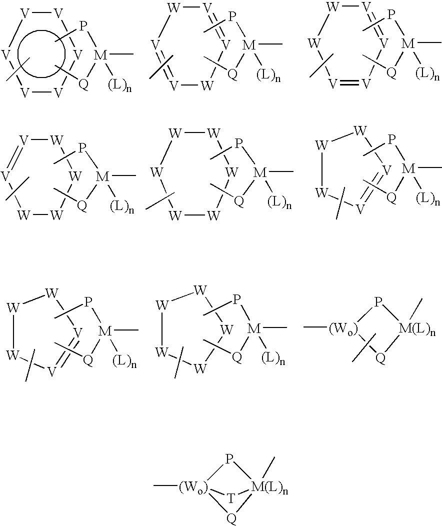
wherein
W is —O—, —S—, —CR¹R¹′—, —C(═O)—, —C(═S)—, —C(═NR² or —NR¹—;
V is —N═, —CR¹═;
P, Q and T are independently absent or are independently chosen from —CR¹R¹′—, —NR¹—, —O—, —S— or —PR¹—;
M is B, Si or Sn;
L is C₁-C₆ alkyl, -Aryl or —F;
n is 1 or 2; o is an integer between 2 and 10.

3. A compound according to claim 1 wherein, the carrier is selected from the group consisting of: 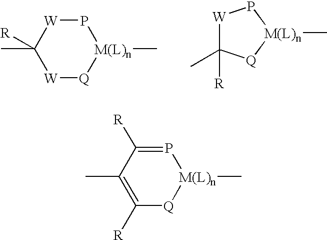
wherein
W is —CR¹R¹′—, —C(═O)—, —C(═S)—, —C(═NR²)— or —NR¹—;
P and Q are independently chosen from —CR¹R¹′—, —NR¹—, —O—, —S—or —PR¹—;
M is B, Si or Sn;
L is C₁-C₆ alkyl, -Aryl or —F;
n is 1 or 2.

4. A compound according to claim 1 wherein the Spacer is a valence bond, C₁-C₆ alkylene-A-, C₂-C₆ alkenylene-A-, C₂-C₆ alkynylene-A-, or 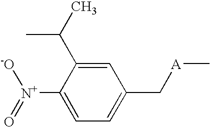
said spacer optionally being connected through A to a linker selected from 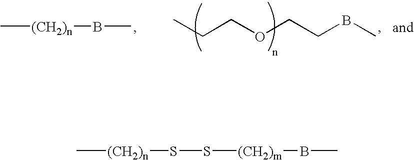
where A is a valence bodn, —C(O)N—, —N—, —O—, —S—, or —C(O)—O—; B is a valence bond, —O—, —S—, —N— or —C(O)N— and connects to S—C-connecting group; R⁸ is selected independently from H, C₁-C₆ alkyl, C₃-C₇ cycloalkyl, aryl or C₁-C₆ alkylene-aryl and n and m independently are integers ranging from 1 to 10.

5. A compound according to claim 1 wherein the S—C-connecting group is a valence bond, —NH—C (═O)—, —NH—C (═O)—C₁-C₆ alkylene-, —S—S—, —S—S—C₁-C₆ alkylene-, —C (═O)—NH—, —C (═O)—NH— (C₁-C₆ alkylene)-, 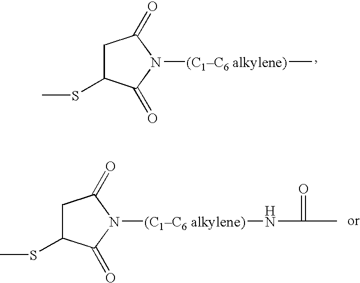
—NH—C(═O)-Arylene-C( )₂—NH—C(═O)—.

6. A compound according to claim 1 wherein, the carrier is -Aryl-B(L)₂— where L is independently chosen from aryl or —F.

7. A compound according to claim 1 where Complementing element is a nucleic acid.

8. A compound according to claim 1 where Complementing element is a sequence of nucleotides selected from the group consisting of DNA, RNA, LNA, PNA, and morpholino derivatives.

9. A library of compounds according to claim 1, wherein each different member of the library comprises a complementing element having a unique sequence of nucleotides, which identifies the functional entity.

10. A method for transferring a functional entity to a recipient reactive group, comprising the steps of
providing one or more building blocks according to claim 1,
contacting the one or more building blocks with a corresponding encoding element associated with a recipient reactive group under conditions which allow for a recognition between the one or more complementing elements and the encoding elements, said contacting being performed prior to, simultaneously with, or subsequent to a transfer of the functional entity to the recipient reactive group.

11. The method according to claim 10, wherein the encoding element comprises one or more encoding sequences comprised of 1 to 50 nucleotides and the one or more complementing elements comprises a sequence of nucleotides complementary to one or more of the encoding sequences.

12. The method of claim 10, wherein the recipient reactive group is an aromatic halogen substituent selected from the group consisting of Br and I, which may be part of a chemical scaffold, and the activating catalyst contains palladium.
